(19) United States
(12) Patent Application Publication
(10) Pub. No.: US 2012/0164025 A1
(43) Pub. Date: Jun. 28, 2012

(54) Disinfecting Package and Methods of Making and Using the Same

(75) Inventors: H. Walker Stockley, III, Spartanburg, SC (US); Michael D. Grah, Greenville, SC (US); Elizabeth R. Gooden, Greenville, SC (US); Denning R. Saum, Greenville, SC (US); Charles E. Marshall, III, Spartanburg, SC (US); William P. Roberts, Spartanburg, SC (US); Stephen F. Compton, Spartanburg, SC (US)

(73) Assignee: Sealed Air Corporation (US), Elmwood Park, NJ (US)

(21) Appl. No.: 13/315,373

(22) Filed: Dec. 9, 2011

Related U.S. Application Data
(60) Provisional application No. 61/421,338, filed on Dec. 9, 2010.

(51) Int. Cl. A61L 9/12

(52) U.S. Cl. 422/29; 422/305; 422/119

(57) ABSTRACT

The presently disclosed subject matter provides a package and methods for disinfecting a wide variety of microbiologically contaminated products. Particularly, the disclosed package comprises a package to house the product to be disinfected and a disinfecting gas generator positioned within the interior of the package. The materials used to construct the package and/or the gas generator comprises an active agent that neutralizes the disinfecting gas from the interior of the package. The reactivity of the active agent is balanced such that the rate of neutralizing is lower than the rate of disinfecting gas release. As a result, the disinfecting gas has time to function fully before it is neutralized. Thus, when a user opens the package at the end of the disinfecting period, exposure to the disinfecting gas is minimized or eliminated.

### CROSS REFERENCE TO RELATED APPLICATIONS

The subject application claims priority to U.S. Provisional Patent Application No. 61/421,338, filed Dec. 9, 2010, the entire content of which is hereby incorporated by reference herein.

### FIELD OF THE INVENTION

The presently disclosed subject matter relates generally to packages and methods of using such packages to safely and effectively disinfect a product.

### BACKGROUND

The use of a disinfecting gas for retarding, controlling, killing, and/or preventing microbiological contamination of a product is known. Such gases generally include chlorine dioxide, sulfur dioxide, nitrogen dioxide, and the like. However, such disinfecting gases cannot be transported commercially. Rather, disinfection requires the use of a dedicated facility and equipment to supply the disinfecting gas. The equipment takes up space and represents a significant added expense. In addition, controlling the amount of gas generated, the efficiency of the gas production, and the duration of the gas generation has proven difficult and/or unsuccessful using such equipment.

Continuing, disinfecting gas can be toxic to humans. For example, chlorine dioxide gas can be toxic at concentrations greater than 1,000 parts per million (“ppm”). As a result, conventional methods of on-site manufacture of disinfecting gas require not only expensive equipment, but also high levels of operator skill to avoid exposure.

Accordingly, there remains a need for a reliable disinfecting system that does not require dedicated equipment or personnel. In addition, there remains a need for a system in which the disinfecting conditions are supplied from within the interior of a sealed pouch or other container. Further, there remains a need in the art for a system that minimizes or eliminates the risk of user exposure to harmful disinfecting gases. Such a system would offer increased mobility, field use, and would offer an increased safety benefit to users.

### SUMMARY

In some embodiments, the presently disclosed subject matter is directed to a container for disinfecting a product. Particularly, the container comprises a package having an interior and a sealable opening through which a product can be placed into the interior of the package. The container also comprises a disinfecting gas generator disposed within the interior of the package. The package comprises a pair of opposing films joined together along a pair of opposing sides and a bottom bridging the sides. The package also comprises at least one of the following: (1) at least one of the package films comprises a neutralizing layer and an active agent carried by the neutralizing layer, (2) an active agent housed in the disinfecting gas generator, or (3) both (1) and (2).

In some embodiments, the presently disclosed subject matter is directed to a method of disinfecting a product. Particularly, the method comprises supplying a product to be disinfected, supplying a package having an interior and a sealable opening through which said product can be placed into the interior of the package, and supplying a disinfecting gas generator. The method also comprises placing the product and the generator within the interior of the package. The method comprises sealing the package and activating the generator to introduce an amount of disinfecting gas into the interior effective to disinfect the product. The method further comprises maintaining the package in a sealed condition for a disinfecting period and opening the package and removing the disinfected product. The package comprises a pair of opposing films joined together along a pair of opposing sides and a bottom bridging the sides and comprises at least one of the following: (1) at least one of the package films comprises a neutralizing layer and an active agent carried by the neutralizing layer, (2) an active agent housed in the disinfecting gas generator, or (3) both (1) and (2).

In some embodiments, the presently disclosed subject matter is directed to a kit comprising a package having an interior and a sealable opening and a disinfecting gas generator. The package comprises a pair of opposing films joined together along a pair of opposing sides and a bottom bridging the sides. The package also comprises at least one of the following: (1) at least one of the package films comprises a neutralizing layer and an active agent carried by the neutralizing layer, (2) the disinfecting gas generator comprises an active agent, or (3) both (1) and (2).

### BRIEF DESCRIPTION OF THE DRAWINGS

FIGS. 1a and 1b are front elevation views of one embodiment of the disclosed disinfecting package.

FIG. 2a is a top plan view of one embodiment of a package that can be used with the disclosed disinfecting package.

FIG. 2b is a bottom plan view of one embodiment of a package that can be used with the disclosed disinfecting package.

FIG. 3 is front elevation view of one embodiment of a package that can be used with the presently disclosed subject matter.

FIGS. 4a, 4b, and 4c are top plan views of embodiments of disinfecting gas generators that can be used with the presently disclosed subject matter.

FIG. 4d is a fragmentary view of a compartment of a gas generator according to some embodiments of the presently disclosed subject matter.

FIG. 4e is a top plan view of one embodiment of a disinfecting gas generator that can be used in accordance with the presently disclosed subject matter.

FIG. 5a is a top plan view of one embodiment of a receptacle that can be used with the presently disclosed subject matter.

FIG. 5b is a side elevation view of the receptacle of FIG. 5a.

FIG. 5c is a sectional view taken along line 5c-5c of FIG. 5b.

FIGS. 5d and 5e are enlarged fragmentary views of the lid of FIG. 5c.

FIGS. 5f and 5g illustrate the air flow in the receptacle of FIG. 5c in some embodiments.

FIGS. 6a-6e are perspective views of some embodiments of using the disclosed package.

FIG. 7a is a perspective view of the disclosed package in some embodiments of the presently disclosed subject matter.

FIG. 7b is a cross-sectional view of the package of FIG. 7a.

### DETAILED DESCRIPTION

### I. General Considerations

The presently disclosed subject matter provides a system and method for disinfecting a wide variety of microbiologically contaminated products, such as (but not limited to) sports equipment, military gear, medical products, shoes, mattresses, pet products, pillows, furniture, cushions, toys, porous food containers, insect-infested (bed bugs, lice, scabies, etc.) items, and the like. Specifically, in some embodiments, the presently disclosed subject matter is directed to a package that is capable of self-disinfecting. “Self-disinfecting” as used herein refers to packages that have the capability of bringing about disinfecting conditions inside an enclosed package or other sealable container without the necessity of being treated with an externally-supplied sterilizing medium. To this end, in some embodiments, the disclosed packages can be formed from one or more barrier materials such that the disinfecting conditions can be maintained within the package for a desired period of time.

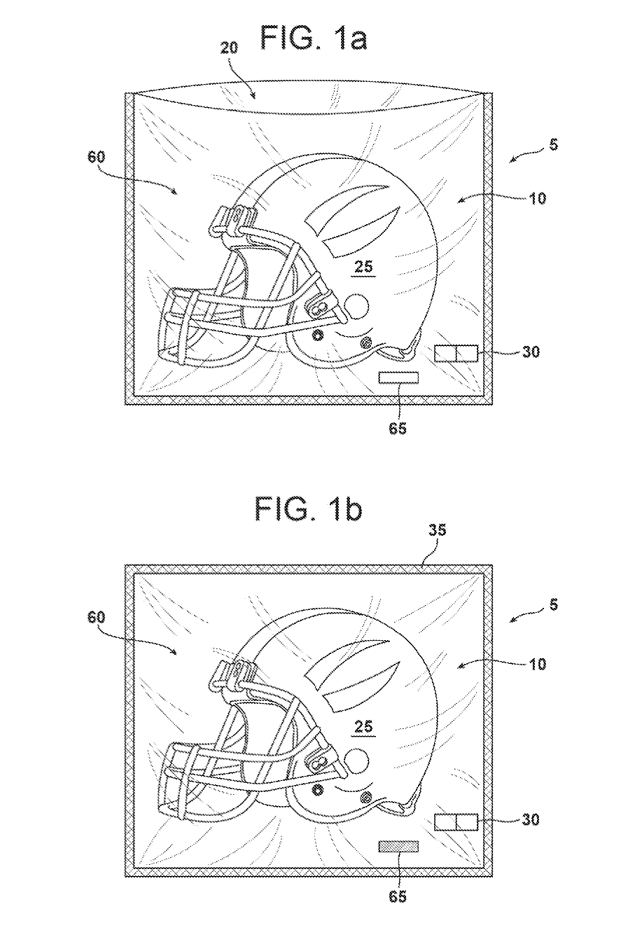

As illustrated in FIG. 1a, in some embodiments, self-disinfecting container 5 can comprise package 10 having interior 60 and sealable opening 20 through which product 25 can be placed into the interior of the package. Disinfecting gas generator 30 (which in some embodiments can be a chlorine dioxide generating sachet) is also disposed within the interior of the package. Optionally, in some embodiments, indicator 65 can be additionally disposed within the interior of package 10 to notify the user when disinfecting conditions have been achieved. Particularly, in some embodiments, indicator 65 is adapted to specify when an effective amount of disinfecting gas has been generated.

Package opening 20 can be sealed, for example, by securing the open overlapping margins to form seal 35, as illustrated in FIG. 1b. Generator 30 is then activated to generate disinfecting gas (such as, but not limited to, chlorine dioxide). The disinfecting gas effectively disinfects product 25 over a period of time. In some embodiments, package 10 and/or generator 30 comprises an active agent that neutralizes the disinfecting gas. The reactivity of the active agent in package 10 can be balanced such that the rate of neutralizing is lower than the rate of disinfecting gas release. As a result, the disinfecting gas has time to function fully before it is neutralized. Thus, when a user opens the package at the end of the disinfecting period, exposure to the disinfecting gas is minimized or eliminated.

### II. DEFINITIONS

While the following terms are believed to be understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” can refer to “one or more” when used in the subject specification, including the claims. Thus, for example, reference to “a package” can include a plurality of such packages, and so forth.

Unless otherwise indicated, all numbers expressing quantities of components, conditions, and so forth used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, and/or percentage can encompass variations of, in some embodiments ±20%, in some embodiments ±10%, in some embodiments ±5%, in some embodiments ±1%, in some embodiments ±0.5%, and in some embodiments to ±0.1%, from the specified amount, as such variations are appropriate in the disclosed materials and methods.

As used herein, the term “abuse layer” can refer to an outer film layer and/or an inner film layer, so long as the film layer serves to resist abrasion, puncture, and other potential causes of reduction of package integrity, as well as potential causes of reduction of package appearance quality. Abuse layers can comprise any polymer, so long as the polymer contributes to achieving an integrity goal and/or an appearance goal. In some embodiments, the abuse layer can comprise polyamide, ethylene/propylene copolymer, and/or combinations thereof.

The term “active agent” as used herein refers to a composition with disinfecting gas neutralizing or deactivating properties. For example, in some embodiments, active agents can include (but are not limited to) scavenging agents that bind and deactivate the disinfecting gas. Alternatively or in addition, in some embodiments, the active agent can comprise a catalyst that breaks down the disinfecting gas into components that react with a scavenging agent.

As used herein, the terms “barrier” and/or “barrier layer” can refer to the ability of a film or film layer to serve as a barrier to one or more gases. For example, oxygen barrier layers can comprise, but are not limited to, ethylene/vinyl alcohol copolymer, polyvinyl chloride, polyvinylidene chloride, polyamide, polyester, polyacrylonitrile, and the like, as known to those of ordinary skill in the art. In some embodiments, the barrier film or layer has an oxygen transmission rate of no more than 100 cc O₂/m²·day·atm; in some embodiments, less than 50 cc O₂/m²·day·atm; in some embodiments, less than 25 cc O₂/m²·day·atm; in some embodiments, less than 10 cc O₂/m²·day·atm; in some embodiments, less than 5 cc O₂/m²·day·atm; and in some embodiments, less than 1 cc O₂/m²·day·atm (tested at 1 mil thick and at 25° C. in accordance with ASTM D3985, herein incorporated by reference in its entirety).

As used herein, the term “bulk layer” can refer to any layer of a film that is present for the purpose of increasing the abuse-resistance, toughness, and/or modulus of a film. In some embodiments, bulk layers can comprise polyolefin, ethylene/alpha-olefin copolymer, ethylene/alpha-olefin copolymer plastomer, low density polyethylene, linear low density polyethylene, and combinations thereof.

As used herein, the term “copolymer” can refer to polymers formed by the polymerization reaction of at least two different monomers. For example, the term “copolymer” can include the copolymerization reaction product of ethylene and an alpha-olefin, such as 1-hexene. However, in some embodiments the term “copolymer” can include, for example, the copolymerization of a mixture of ethylene, propylene, 1-hexene, and 1-octene.

The term “disinfecting” or “disinfected” refers to the process of cleansing so as to destroy and prevent the growth of pathogenic microorganisms. In some embodiments, disinfecting can refer to a combination of a concentration of disinfecting gas and a time exposure interval that will disinfect a product subjected to the gas within a container. Disinfecting conditions can be provided by a wide range of disinfecting gas concentrations in combination with various time intervals. In general, the higher the concentration of a disinfecting gas, the shorter a corresponding time interval is needed to establish disinfecting conditions. Accordingly, the effective amount of a disinfecting gas can vary depending upon the length of exposure of the product to the gas. Further, although disinfecting conditions are described herein, in some embodiments the presently disclosed subject matter can equally be used to sterilize a product. Thus, when the term “disinfecting” is used, there are some embodiments where the term includes both disinfecting and sterilizing.

As used herein, the term “disinfecting gas” refers to a gas that effectively destroys, neutralizes, and/or inhibits the growth of pathogenic microorganisms without adversely affecting the product being disinfected. In some embodiments, disinfecting gas includes (but is not limited to) chlorine dioxide, ethylene oxide, sulfur dioxide, nitrogen dioxide, nitric oxide, nitrous oxide, carbon dioxide, hydrogen sulfide, hydrocyanic acid, dichlorine monoxide, ozone, and the like. However, this list is not exhaustive and disinfecting gases suitable for use with the presently disclosed subject matter can include any gas that is capable of disinfecting a product.

The term “disinfecting gas generator” as used herein is not limited and refers to any device that generates a disinfecting gas. For example, in some embodiments, the generator can comprise two or more reactants that are mixed on demand to produce a disinfecting gas.

The term “disinfecting period” refers to the time period required to disinfect a product using the disclosed package. Thus, in some embodiments, the disinfecting period is the amount of time a product remains sealed within the interior of a package after the generator has been initiated.

As used herein the term “effective amount” can refer to either an amount of disinfecting gas provided to a package or a time during which the disinfecting gas has been provided to a package to achieve disinfecting conditions. As will be recognized, an effective amount of disinfecting gas depends on the relationship between the amount of gas utilized and the time period during which it is utilized. For example, when products are subjected to lengthy periods of disinfection, less amounts of disinfecting gas can be effective in achieving a disinfected product. When products are subjected to large amounts of disinfecting gas, shorter exposure time periods can be appropriate to disinfect such products.

As used herein, the term “film” can include, but is not limited to, a laminate, sheet, web, coating, and/or the like, that can be used to package a product. The film can be a rigid, semi-rigid, or flexible product. In some embodiments, the disclosed film is produced as a fully coextruded film, i.e., all layers of the film emerging from a single die at the same time. In some embodiments, the film is made using a flat cast film production process or a round cast film production process. Alternatively, the film can be made using a blown film process, double bubble process, triple bubble process, and/or adhesive or extrusion coating lamination in some embodiments. Such methods are well known to those of ordinary skill in the art.

The term “frangible” as used herein refers to a membrane or seal that is rupturable or fragile. It should be understood that the term “frangible” can indicate the susceptibility of being broken without implying weakness. Thus, in referring to a frangible seal separating compartments of a sachet, it can be meant that when so sealed the compartments are united together in a fluid impervious manner, and when the seal is broken or severed, the contents of the compartments are free to intermix. Thus, the frangible seal in an intact state serves to maintain the integrity of a product chamber reservoir for maintaining fluid, semi-fluid, and/or solid products therein, but in a broken or severed state allows for passage of these products along a delaminated seal area. Frangible seals are commonly referred to as “easy open seals”, “peelable seals” and/or other similar descriptors by those of ordinary skill in the related art.

As used herein, the term “heat seal” refers to any seal of a first region of a film surface to a second region of a film surface, wherein the seal is formed by heating the regions to at least their respective seal initiation temperatures. Heat-sealing is the process of joining two or more thermoplastic films or sheets by heating areas in contact with each other to the temperature at which fusion occurs, usually aided by pressure. In some embodiments, heat-sealing can be inclusive of thermal sealing, melt-bead sealing, impulse sealing, dielectric sealing, and/or ultrasonic sealing. The heating can be performed by any one or more of a wide variety of means, such as (but not limited to) a heated bar, hot wire, hot air, infrared radiation, ultrasonic sealing, and the like.

The term “interior” as used herein with regard to a container refers to the actual inside portion of the container into which a product is inserted.

As used herein, the term “multilayer film” can refer to a thermoplastic film having one or more layers formed from polymeric or other materials that are bonded together by any conventional or suitable method, including one or more of the following methods: coextrusion, extrusion coating, lamination, vapor deposition coating, solvent coating, emulsion coating, or suspension coating.

The term “neutralize” or “neutralization” as used herein refers to the inactivation of a disinfecting gas to a form that is not harmful and/or toxic to a human.

The term “neutralizing layer” as used herein refers to a film layer with the capacity to render a disinfecting gas (such as chlorine dioxide) harmless or less harmless to humans. For example, in some embodiments, a neutralizing film layer can comprise an active agent that binds to, breaks down, chemically modifies, or otherwise neutralizes a disinfecting gas to a nontoxic form or concentration.

The term “opening” as used herein refers to a portion of the top surface of a container (such as a pouch) that allows a user to access a product housed within the interior volume of the container.

As used herein, the term “package” refers to packaging materials configured around a product being packaged, and can comprise (but is not limited to) bags, pouches, trays, and the like, as well as a disinfecting gas generator. In some embodiments, the package can comprise a product that is surrounded by a packaging material.

As used herein, the term “polymer” can refer to the product of a polymerization reaction, and can be inclusive of homopolymers, copolymers, terpolymers, and the like. In some embodiments, the layers of a film can consist essentially of a single polymer, or can have still additional polymers together therewith, i.e., blended therewith. The term “polymeric” can be used to describe a polymer-containing material (i.e., a polymeric film).

The term “package” as used herein includes a pouch, a bag, or like containers, either pre-made or made at the point of packaging. Thus, in some embodiments, the term “package” encompasses flexible and/or rigid packages made of plastics, metal, and the like.

The term “sachet” as used herein refers to a closed receptacle for housing at least one reactant. The sachet is closed in the sense that the reactants, prior to initiation, are substantially retained within the sachet. Sachets can be constructed of, e.g., gas permeable, dissolvable, and/or liquid permeable materials. In some embodiments, sachets can be frangible to allow for puncturing of the sachets, resulting in the exposure of the reactants contained therein. Alternatively or in addition, in some embodiments, sachets can include one or more frangible seal to allow two or more reactants housed within the sachet to intermix.

As used herein, the term “seal” can refer to any seal of a first region of a film surface to a second region of a film or substrate surface. In some embodiments, the seal can be formed by heating the regions to at least their respective seal initiation temperatures using a heated bar, hot air, infrared radiation, ultrasonic sealing, and the like. In some embodiments, the seal can be formed by an adhesive. Such adhesives are well known in the packaging art. Alternatively or in addition, in some embodiments, the seal can be formed using a UV or e-beam curable adhesive seal.

The term “sealable” as used herein refers to the characteristic of being capable of becoming sealed, i.e., closed with a means that must be removed or broken to gain access.

As used herein, the terms “seal layer”, “sealing layer”, “heat seal layer”, and/or “sealant layer” refer to an outer film layer or layers involved in heat sealing of the film to itself, another film layer of the same or another film, and/or another product that is not a film. Heat sealing can be performed by any one or more of a wide variety of manners known to those of ordinary skill in art, including using heat seal technique (e.g., melt-bead sealing, thermal sealing, impulse sealing, ultrasonic sealing, hot air, hot wire, infrared radiation, and the like), adhesive sealing, UV-curable adhesive sealing, and the like.

The term “sterilizing conditions” refers to a combination of a concentration of disinfecting gas and a time exposure interval that will sterilize a product that is subjected to the disinfecting gas within a package. Sterilizing conditions can be provided by a wide range of disinfecting gas concentrations in combination with various time intervals. In general, the higher the concentration of a disinfecting gas, the shorter a corresponding time interval is needed to establish sterilizing conditions. Accordingly, the effective amount of a disinfecting gas can vary depending upon the length of exposure of the product to the disinfecting gas.

As used herein, the term “tie layer” can refer to any internal film layer having the primary purpose of adhering two layers to one another. In some embodiments, the tie layers can comprise any nonpolar polymer having a polar group grafted thereon, such that the polymer is capable of covalent bonding to polar polymers such as polyamide and ethylene/vinyl alcohol copolymer. In some embodiments, the tie layers can comprise, but are not limited to, modified polyolefin, modified ethylene/vinyl acetate copolymer, and/or homogeneous ethylene/alpha-olefin copolymer.

All compositional percentages used herein are presented on a “by weight” basis, unless designated otherwise.

### III. Package 10

### III.A. Generally

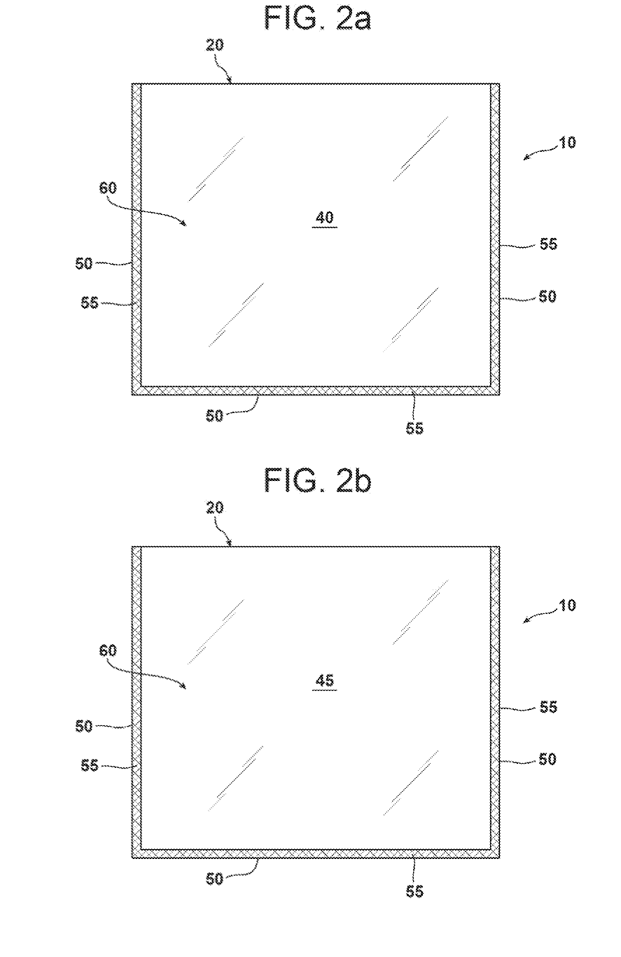

As set forth above, container 5 comprises package 10, having interior 60 and sealable opening 20 through which a product can be placed into the interior of the package. In some embodiments package 10 can comprise a pair of opposing films joined together along a pair of opposing sides and a bottom bridging the side. Particularly, as illustrated in FIGS. 2a and 2b, package 10 can be constructed from upper and lower packaging films 40, 45 that have been sealed at side and bottom edges 50 to create seals 55. Seals 55 can be formed using any of a wide variety of methods known in the art, including (but not limited to) adhesive, thermal bonds, ultrasonic bonds, radio frequency sealing, and the like. Thus, together films 40, 45 define a package with interior 60 to house a product to be disinfected. As would be apparent to those of ordinary skill in the art, in some embodiments, package 10 can alternatively be formed from a single film that has been center folded at one edge.

The top edge of package 10 can be unsealed to form opening 20. Opening 20 provides a means through which product 25 and/or generator 30 can be inserted into interior 60 of package 10. Likewise, in some embodiments, after disinfection has occurred, product 25 and/or generator 30 can be removed from the inner compartment of package 10 via opening 20. In some embodiments opening 20 is reclosable to allow for multiple opening and closing actions of the package. In such embodiments, package 10 can include any of a wide variety of reclosable elements known in the art including (but not limited to) adhesives, mated tracks, mated dimples, threaded lids, caps, clamps, o-rings, gaskets, VELCRO®, zippers, and/or combinations thereof. The reclosable elements can be opened and closed by applying finger pressure or by using an auxiliary device, such as a slider. Such methods and elements are well known to those of ordinary skill in the art. In some embodiments, opening 20 can include a tamper-evident closure to indicate that tampering and/or premature opening of the package has occurred. Such tamper-evident closures are well known in the art and can include (but are not limited to) destructive adhesive tape or film closures, clamps, caps, removable tear strips, and the like.

Films 40, 45 can have any total thickness so long as the film provides the desired properties for the particular packaging operation in which it is to be used. Nevertheless, in some embodiments the disclosed film has a total thickness of from about 0.1 mil to about 20 mils; in some embodiments, from about 0.2 mil to about 10 mils; in some embodiments, from about 0.3 mils to about 5.0 mils; and in some embodiments, from 1.0 to 3.0 mils.

Generally, films 40, 45 can be multilayer or monolayer. Typically, however, the films employed will have two or more layers to incorporate a variety of properties, such as, for example, sealability, gas impermeability, and toughness into a single film. Thus, in some embodiments, films 40, 45 can comprise a total of from about 1 to about 20 layers; in some embodiments, from about 4 to about 12 layers; and in some embodiments, from about 5 to about 9 layers. Accordingly, the disclosed film can comprise 1, 2, 3, 4, 5, 6, 7, 8, 9, 10, 11, 12, 13, 14, 15, 16, 17, 18, 19, or 20 layers. One of ordinary skill in the art would also recognize that films 40, 45 can comprise more than 20 layers, such as in embodiments wherein the films comprise microlayering technology.

Films 40, 45 can be provided in sheet or film form and can be any of the films commonly used for the disclosed type of packaging. To this end, films 40, 45 can be constructed by any suitable process known to those of ordinary skill in the art, including (but not limited to) coextrusion, lamination, extrusion coating, and combinations thereof. See, for example, U.S. Pat. No. 6,769,227 to Mumpower, the content of which is herein incorporated by reference in its entirety.

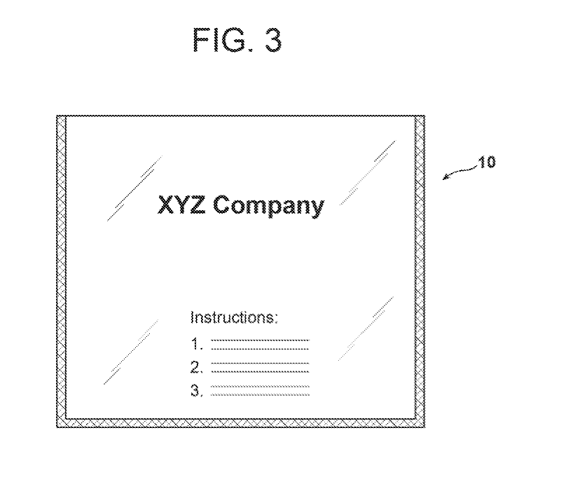

In some embodiments, films 40, 45 can comprise printed information such as (but not limited to) product size, type, name of manufacturer, instructions for use, and the like, as illustrated in FIG. 3. Such printing methods are well known to those of ordinary skill in the packaging art.

In some embodiments, films 40, 45 can be transparent (at least in the non-printed regions) such that product 25 is at least partially visible through the films. The term “transparent” as used herein can refer to the ability of a material to transmit incident light with negligible scattering and little absorption, enabling objects to be seen clearly through the material under typical unaided viewing conditions (i.e., the expected use conditions of the material). The transparency of films 40, 45 can be at least about any of the following values: 20%, 25%, 30%, 40%, 50%, 65%, 70%, 75%, 80%, 85%, and 95%, as measured in accordance with ASTM D1746.

Although rectangular in the figures, the shape of package 10 is not limited and can have any desired configuration, e.g., rectangular, round, oval, and the like. To this end, package 10 is also not limited in size and can include dimensions to house a wide variety of products. Further, package 10 is not restricted and can be a flexible, semi-flexible, rigid, semi-rigid, collapsible, and/or lidded container, as can be appreciated by those of ordinary skill in the art.

### III.B. Neutralizing Layer

As set forth herein above, the films used to construct package 10 can comprise an active agent that neutralizes the disinfecting gas produced by generator 30. For example, in some embodiments, the active agent can comprise scavenging agents that bind and deactivate the disinfecting gas produced by generator 30. Such scavenging agents can include (but are not limited to) metals, metal oxides, sulfite salts, bisulfite salts, sulfide salts, zeolites, substituted phenols, lignin, substituted pyrroles, substituted thiophenes, hydroquinones, antioxidants, and the like. In some embodiments, the active agent can comprise a catalyst that breaks down the disinfecting gas into components that react with a scavenging agent. One of ordinary skill in the art would understand that the above examples are not exhaustive and the disclosed active agent can include any agent that neutralizes a disinfecting gas.

The active agent can be incorporated into or onto films 40, 45 using any of a variety of suitable techniques known in the art. For example, the active agent can be coextruded into a layer by blending into the web material. To this end, the layer comprising the active agent can be termed a “neutralizing layer.” Thus, in some embodiments, at least one of films 40, 45 comprises a neutralizing layer and an active agent carried by the neutralizing layer.

In some embodiments, the neutralizing layer can be formulated to include varying levels of active agent. Particularly, in some embodiments, the active agent can be present in the range of about 0.1% to about 80% (by weight) of the film layer. In some embodiments, the active agent can be present in the range of about 5% to about 30% (by weight) of the layer. Thus, in some embodiments, the active agent can be present in films 40 and/or 45 in the range of about 5, 6, 7, 8, 9, 10, 11, 12, 13, 14, 15, 16, 17, 18, 19, 20, 21, 22, 23, 24, 25, 26, 27, 28, 29, or 30%, based on the total weight of the neutralizing layer. One of ordinary skill in the art would appreciate that the ranges set forth above can be varied, depending on film thickness and the particular active agent used.

The neutralizing layer can be any layer of films 40, 45 of package 10. For example, in some embodiments the neutralizing layer can be positioned adjacent to a permeable layer to facilitate migration of the disinfecting gas into the neutralizing layer where it contacts the active agent and is neutralized. Alternatively or in addition, in some embodiments, the neutralizing layer can be a sealant layer of films 40 and/or 45.

Alternatively or in addition to providing the active agent in a layer of the films, the presently disclosed subject matter also includes embodiments wherein the active agent is applied as a coating to an interior surface of films 40 and/or 45 (i.e., package interior 60). To this end, the coating can be applied using coating or printing technology, such as (but not limited to) gravure coating or printing, roll coating, lithographic coating or printing, and/or spraying. Such technology is well known to those of ordinary skill in the art. In some embodiments the active agent can be incorporated into a free standing film, sachet, or other device that is positioned within the interior of package 10 prior to sealing the package, as set forth in more detail herein below.

### III.C. Barrier Layer

Films 40, 45 can comprise one or more barrier layers to prevent the escape of disinfecting gas from the interior of the package. Such barrier layers can comprise, for example, polymerized ethylene vinyl alcohol, polyvinyl chloride, polyvinylidene chloride, polyamide, polyester, polyacrylonitrile, and the like, as known to those of ordinary skill in the packaging art.

However, such a barrier layer is not required. Particularly, to promote active transport of the disinfecting gas, in some embodiments films 40, 45 can comprise varying degrees of permeability. As such, films 40, 45 can provide a metered rate of neutralizing the disinfecting gas via contact with the neutralizing agent in the film structure. Thus, elimination of the barrier layers from films 40 and/or 45 can be beneficial when it is desired to control the rate of permeability and rate of neutralization of the disinfecting gas. Polymer components used to fabricate permeable films can include (but are not limited to) high density polyethylene, low density polyethylene, linear low density polyethylene, ethylene/vinyl acetate copolymer, ethylene/alpha-olefin copolymer, propylene/ethylene copolymer, and combinations thereof. To this end, films 40, 45 can comprise multiple layers of polymers or polymer blends to achieve a desired permeability of disinfecting gas. Permeability of polymers to chlorine dioxide is similar to the high-to-low range of “oxygen transmission rate” of polymers. Such routine experimentation is within the knowledge of one of ordinary skill in the art. See, for example, “Mass Transfer Study of Chlorine Dioxide Gas Through Polymeric Packaging Materials”, Netrami, S., Rubino, M., Auras, R., and Annous, B. A, May 2009, School of Packaging, Michigan State University, the entire disclosure of which is hereby incorporated by reference.

### III.D. Other Layers

In some embodiments, films 40, 45 used to construct package 10 can comprise an abuse layer. As used herein, the term “abuse layer” refers to an outer film layer and/or an inner film layer, so long as the film layer serves to resist abrasion, puncture, and other potential causes of reduction of package integrity, as well as potential causes of reduction of package appearance quality. Abuse layers can comprise any polymer, so long as the polymer contributes to achieving an integrity goal and/or an appearance goal. Thus, in some embodiments, an abuse layer can comprise (but is not limited to) ethylene/alpha-olefin copolymer, propylene/ethylene copolymer, polyamide, ethylene/vinyl acetate copolymer, ethylene/methyl acrylate copolymer, and ethylene/butyl acrylate copolymer, and the like, as known to those of ordinary skill in the art.

In some embodiments, films 40, 45 can include one or more tie layers. As used herein, the term “tie layer” refers to any internal layer having the primary purpose of adhering two layers to one another. In some embodiments, tie layers can comprise any polymer having a polar group grafted thereon, polyolefin, modified polyolefin, ethylene/vinyl acetate copolymer, modified ethylene/vinyl acetate copolymer, and homogeneous ethylene/alpha-olefin copolymer.

In some embodiments, films 40, 45 can include one or more bulk layers. The term “bulk layer” refers to any layer of a film that is present for the purpose of increasing the abuse-resistance, toughness, modulus, etc. of a multilayer film. Bulk layers generally comprise polymers that are inexpensive relative to other polymers in the film that provide some specific purpose unrelated to abuse-resistance, modulus, etc.

The polymer components used to fabricate films 40, 45 can also comprise appropriate amounts of other additives normally included in such compositions. For example, slip agents (such as talc), antioxidants, fillers, dyes, pigments and dyes, radiation stabilizers, antistatic agents, elastomers, and the like can be added to the disclosed films. See, for example, U.S. Pat. Nos. 7,205,040 to Peiffer et al.; 7,160,378 to Eadie et al.; 7,160,604 to Ginossatis; 6,472,081 to Tsai et al.; 6,222,261 to Horn et al.; 6,221,470 to Ciacca et al.; 5,591,520 to Migliorini et al.; and 5,061,534 to Blemberg et al., the disclosures of which are hereby incorporated by reference in their entireties.

### IV. Disinfecting Gas Generator 30

### IV.A. Generally

As set forth above, container 5 comprises disinfecting gas generator 30 disposed within the interior of package 10. It should be noted that disinfecting gas generator 30 is not limited and can include any device that generates a disinfecting gas. Such disinfecting gases can include (but are not limited to) chlorine dioxide, ethylene oxide, sulfur dioxide, nitrogen dioxide, nitric oxide, nitrous oxide, carbon dioxide, hydrogen sulfide, hydrocyanic acid, dichlorine monoxide, ozone, and the like.

For example, in some embodiments, generator 30 can comprise two or more reactants 80, 85 that are mixed to produce a disinfecting gas. Particularly, first reactant 80 can be an aqueous acidic solution, such as (but not limited to) aqueous solutions of citric acid, hydrochloric acid, sulfuric acid, phosphoric acid, acetic acid, or combinations thereof. In some embodiments, second reactant 85 can be a metal chlorite salt, such as (but not limited to) sodium chlorite. One of ordinary skill in the art would appreciate that first and second reactants 80, 85 are not limited and can include any reactants that can intermix to produce a disinfecting gas (such as chlorine dioxide). It should be understood that the disclosed apparatus and methods are readily applicable to the delivery of more than one disinfecting gas at one time. For example, in some embodiments, gas generator 30 can generate both chlorine dioxide and sulfur dioxide.

In some embodiments, second reactant 85 can comprise an inert particulate (such as silica) to aid in minimizing free liquid upon mixing with first reactant 80. The inert particulate can also contribute to the gas-liquid and liquid-solid surface area content of the co-reactant mixture to facilitate vaporization of the disinfecting gas. In some embodiments, the inert particulate is present in an amount of from about 1 part reactant to about 4 about parts inert particulate.

The geometry and size of generator 30 can be adapted to suit various parameters, including the amount and type of disinfecting gas to be used, the desired surface area of the product to be disinfected, and the like. Such adaptations can be accomplished through routine experimentation, as would be apparent to those of ordinary skill in the art.

### IV.B. First Embodiment of Sachet 70

In some embodiments, disinfecting gas generator 30 can comprise a sachet housing reactants 80, 85 and can be positioned within the interior of package 10. Sachet 70 can be constructed from any of a wide variety of materials. For example, in some embodiments, sachet 70 can be constructed from air permeable materials (such as, but not limited to, perforated Tyvek®, non woven fabric, filter material, and the like), air impermeable materials, or combinations thereof.

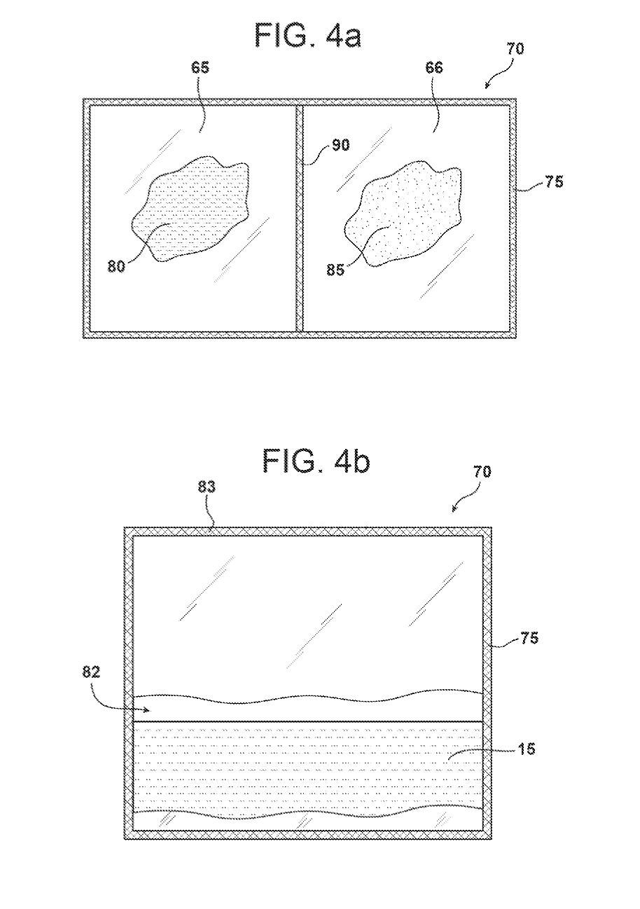

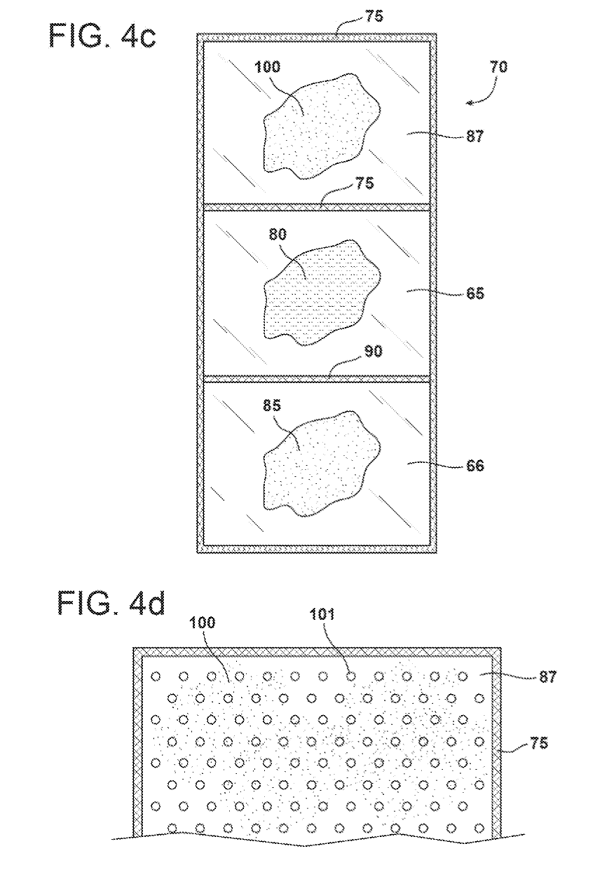

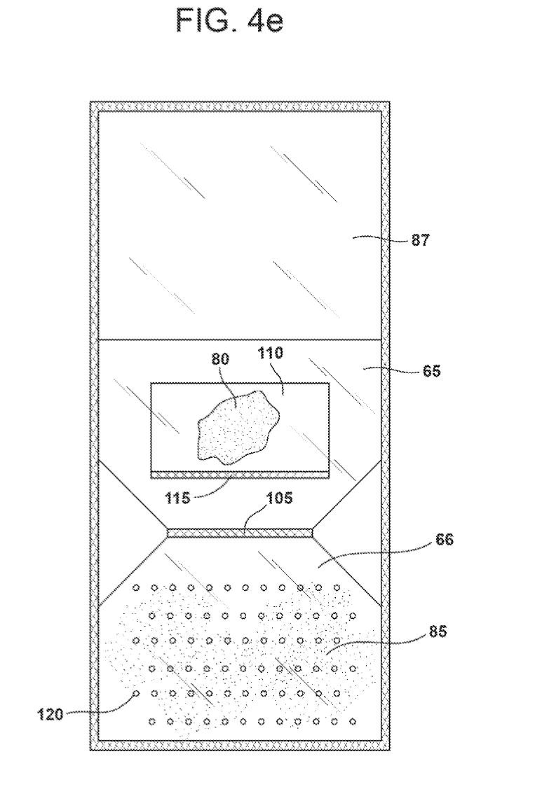

As illustrated in FIG. 4a, sachet 70 can be sealed around the edges by perimeter seal 75 to form at least two compartments 65, 66. In some embodiments, compartments 65, 66 can be separated by frangible seal 90. Reactants 80, 85 can be housed within sachet compartments 65, 66. Thus, when the frangible seal is ruptured, reactants 80, 85 can combine to generate a disinfecting gas. In some embodiments, at least a portion of the materials used to construct sachet 70 is permeable to allow the generated disinfecting gas to escape the sachet. Such sachets are well known in the art. See, for example, U.S. Pat. Nos. 6,607,696 and 6,294,108, the entire disclosures of which are hereby incorporated by reference. One of ordinary skill in the art would appreciate that the disclosed generator is not limited to the sachet of FIG. 4a and can include any device that generates a disinfecting gas on demand.

### IV.C. Second Embodiment of Sachet 70

Alternatively, as illustrated in FIG. 4b, reactants 80, 85 can be combined into mixture 15 and then placed into interior 82 of sachet 70. In some embodiments, reactants 80, 85 can be added to interior 82 of sachet 70 and then combined to form mixture 15. Sachet 70 can then be sealed using closure 83, which can include any of a wide variety of closure means, including (but not limited to) adhesives, mated tracks, mated dimples, threaded lids, caps, clamps, o-rings, gaskets, VELCRO®, zippers, and/or combinations thereof. In such embodiments, sachet 70 can be constructed at least partially from a non-woven, breathable material (such as, for example, Tyvek®) to allow the generated disinfecting gas to diffuse through the breathable material. In some embodiments, sachet 70 can comprise the breathable material on one face and a polymeric (non-breathable) material on an alternate face.

### IV.D. Third Embodiment of Sachet 70

In some embodiments, sachet 70 can comprise a compartment for housing active agent 100. Particularly, as illustrated in FIG. 4c, sachet 70 can comprise first and second compartments 65, 66 for housing first and second reactants 80, 85, as well as third compartment 87 for housing active agent 100. In some embodiments, first and second compartments can be separated by frangible seal 90 or can be contiguous (as illustrated in FIG. 4e). The active agent can be a material that scavenges the disinfecting gas after disinfection/sterilization has occurred. Suitable active agents can include (but are not limited to) sulfite salt, ascorbate salt, ascorbic acid, phenolic compounds, phenolic antioxidants, activated carbon, or combinations thereof. Active agent 100 can be a loose solid housed within third compartment 87, or can be impregnated into a porous substrate, such as a carbon-impregnated air filter pad. As illustrated in FIG. 4d, in some embodiments, third compartment 87 comprises one or more apertures 101 to allow the generated gas to contact active agent 100. In some embodiments, apertures 101 are provided in a spaced apart relation along one or both sides of third compartment 87. Such apertures are not limited and can include any of a wide variety of openings, holes, slits, gaps, or any shaped passage through which a gas can enter or exit third compartment 87.

As illustrated in FIG. 4e, in some embodiments, at least one of first or second reactants 80, 85 can be housed within inner package 110 comprising frangible seal 115. For example, in some embodiments, an aqueous acidic reactant can be housed within inner package 110. As used herein, the term “frangible seal” refers to a seal that is sufficiently durable to allow normal handling of the package but will rupture or substantially rupture under pressure applied by manipulating inner package 110. Thus, as would be apparent to those of ordinary skill in the art, frangible seal 115 can be ruptured by physically or mechanically manipulating the inner package. Frangible seal 115 can be formed using any of a number of well known methods. For example, in some embodiments, the frangible seal can be formed using a seal bar heated to a cooler temperature than used to form typical package perimeter seals. Frangible seals are known to those of ordinary skill in the art. See, for example U.S. Pat. No. 6,983,839 to Bertram et al. and U.S. Patent Application Publication No. 2006/0093765 to Mueller, the entire disclosures of which are incorporated by reference herein.

As illustrated in FIG. 4e, first and second compartments 65, 66 can be contiguous and separated by selectively permeable barrier 105. In some embodiments, selectively permeable barrier 105 can be a liquid-selective barrier such that when first reagent 80 is released from inner package 110, the reagent flows into second compartment 66 to contact second reagent 85. The term “selectively permeable” as used herein refers to an entity that allows passage of certain species (such as liquid) but acts as a barrier to others. Such selectively permeable barrier can be constructed from any of a wide variety of materials, such as (but not limited to) flexible polymeric material, polyethylene, polypropylene, fabric, paper, cellulose, fibrous materials, and the like.

As illustrated in FIG. 4e, in some embodiments, second compartment 66 can be spot sealed together to keep second reactant 85 evenly distributed throughout the compartment. Spot seals 120 can be constructed using any of a wide variety of methods, including (but not limited to) the use of adhesive bonding, thermal welding, ultrasonic welding, pressure bonding, through the use of a solvent, or by any other technique known to those of ordinary skill in the packaging art. Such spot seals are optional and the presently disclosed subject matter includes embodiments wherein sachet 70 does not include such spot seals.

### IV.E. Receptacle 125

In some embodiments, gas generator 30 can be positioned within a receptacle housed within the interior of package 10. Receptacle 125 can take any of a wide variety of shapes and forms, so long as it is capable of holding and/or properly positioning a disinfecting gas generating device (such as sachet 70) within a package. Receptacle 125 can be constructed from any of a wide variety of materials, including (but not limited to) plastic (such as polypropylenes, polyethylenes, polyesters, ABS, polystyrene, vinyl), metal (include pure metals, metal alloys intermetallic compositions and the like), wood, paperboard, chipboard, cardboard, rubber, ceramic materials, and/or any other suitable material.

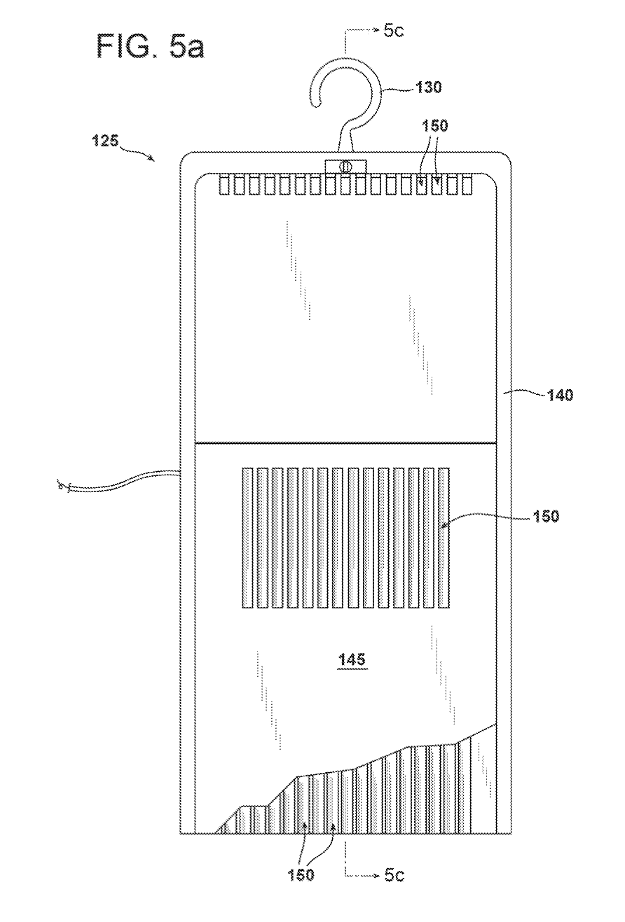

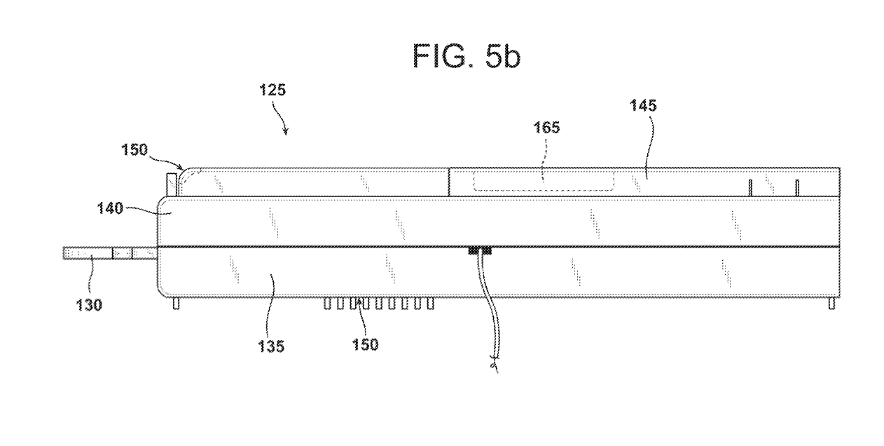

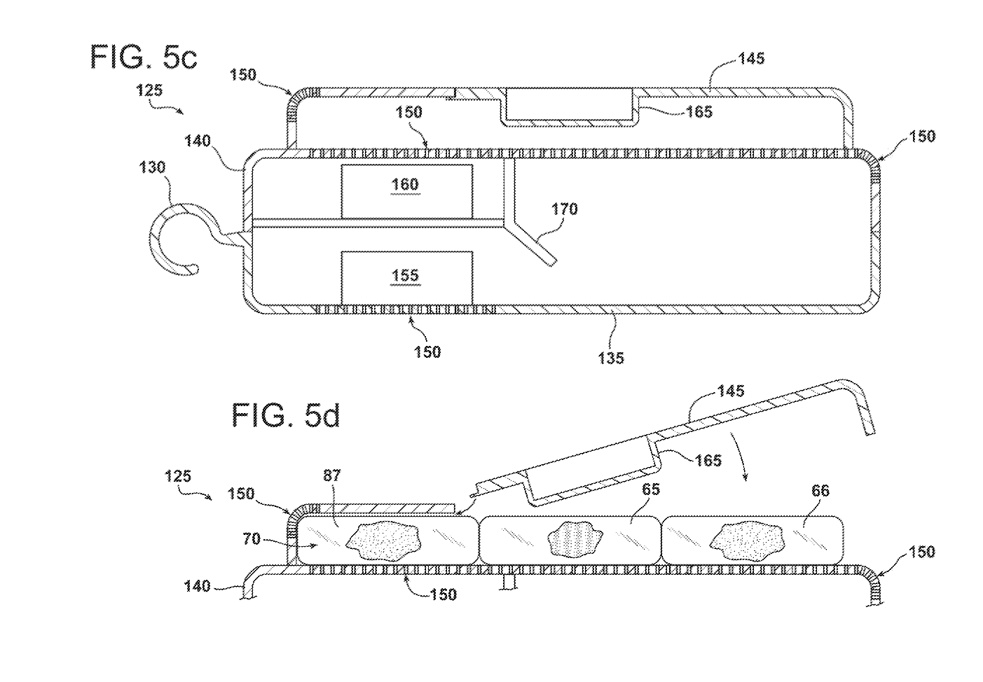

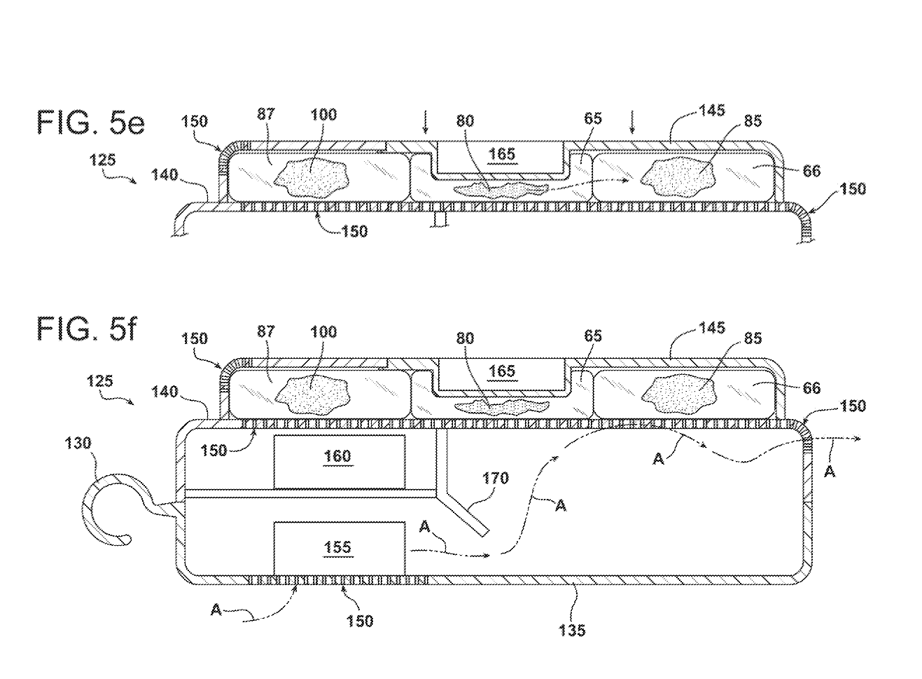

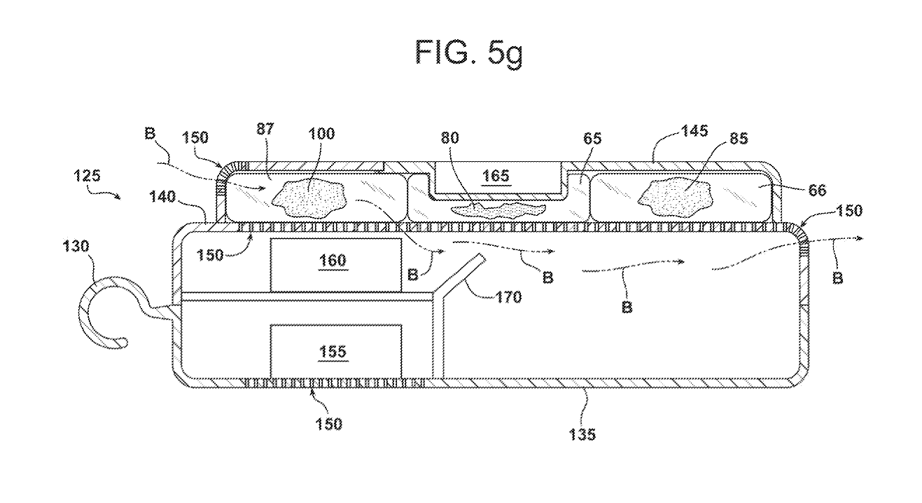

FIGS. 5a and 5b illustrate one embodiment of a receptacle suitable for use with the presently disclosed subject matter. For example, in some embodiments, receptacle 125 can comprise at least one optional support 130 that functions to properly position the receptacle within the interior of package 10. Support 130 that can include any of a wide variety of reinforcements that allow the receptacle to hang or be otherwise properly positioned. For example, in some embodiments, support 130 can include one or more hooks, clips, clasps, hanging loops, cords, fasteners, suction devices, screws, bolts, adhesives, and the like.

Continuing, in some embodiments, receptacle 125 can comprise lower member 135, upper member 140, and lid 145. The upper member, lower member and lid of the receptacle cooperate with each other using an interlocking mechanism, a snap fit arrangement, an adhesive, a magnetic coupling, and/or the like to come together and form the receptacle of FIGS. 5a and 5b. In some embodiments, lower member 135, upper member 140, and lid 145 can comprise vents 150 to permit air flow into and out of the interior of the receptacle.

As illustrated in FIG. 5c, in some embodiments lower member 135 can comprise first fan 155 adjacent to the openings of vent 150 to permit air flow from the interior of the package into the interior of the receptacle. In some embodiments, receptacle 125 can comprise second fan 160 to permit air flow from the inside of the package into the interior of the receptacle. Thus, fans 155, 160 cooperate with vents 150 to direct air flow into and out of the receptacle. Fans 155, 160 and their mounting arrangements are of a conventional construction and thus are not discussed in greater detail herein. In fact, any of a wide variety of fans or airflow generating devices can be readily employed in the presently disclosed subject matter.

Receptacle 125 is also configured to house sachet 70. Particularly, in some embodiments, sachet 70 can be positioned between lid 145 and upper member 140, as illustrated in FIG. 5d. Thus, lid 145 can include enlarged segment 165 positioned adjacent to first sachet compartment 65. In embodiments wherein first sachet compartment 65 comprises inner package 110 housing a reagent (as shown in FIG. 4e), the enlarged segment functions to rupture frangible seal 115 of the inner package to allow reagents 65, 66 to intermix and generate a disinfecting gas, as illustrated in FIG. 5e. In some embodiments, intermixing of the first and second reagents is facilitated when receptacle 125 is hung by support 130, thereby allowing first reagent 65 to flow via gravity and contact second reagent 66. However, support 130 is optional and the presently disclosed subject matter includes embodiments wherein receptacle 125 and/or generator 30 does make use of gravity for intermixing.

Thus, in some embodiments, receptacle 125 comprises first fan 155 that draws air from inside package 10 into the interior of the receptacle via openings 150 in lower member 135. The air is then directed past the sachet compartments where the disinfecting gas that has been generated is carried out of the receptacle and into the interior of package 10, as illustrated in FIG. 5f by Arrows A. In embodiments wherein receptacle 125 comprises second fan 160, air containing the disinfecting gas is drawn from the inside of the package via the second fan and flows into the interior of the receptacle via openings 150 to contact third compartment 87 of sachet 70 that houses active agent 100, as illustrated by Arrows B in FIG. 5g. After contacting the active agent, fan 160 moves the air out of the receptacle through openings 150.

In some embodiments, air flow within receptacle 125 can be controllably restricted (e.g., with adjustable valves, flappers, slats, or other flow restriction elements). To this end, in some embodiments, receptacle 125 can comprise valve 170 to prevent backflow of air from first fan 155 to second fan 160. Any of a wide variety of valves can be incorporated into receptacle 125, including (but not limited to) flapper valves, solenoid valves, wedges, throttle valves, closer springs, ball valves, retainer valves, and the like. One of ordinary skill in the art would recognize that valve 170 is optional and that the presently disclosed subject matter includes embodiments without such a feature.

### V. Indicator 65

In some embodiments, container 5 can comprise indicator 65. Particularly, the indicator can be placed into the interior of package 10 to provide a visible indication upon exposure to an effective amount of disinfecting gas. For example, when chlorine dioxide is employed as the disinfecting gas, indicator 65 can be a chlorine indicator strip that detects the presence of chlorine dioxide gas, as illustrated in FIGS. 1a and 1b. Although various types of indicators can be employed, most common indicators undergo a modification in the presence of a disinfecting gas that results in a visual change. It is also possible to provide a graduated series of color indicators that change in color and intensity as the level of exposure to disinfecting gas increases.

One of ordinary skill in the art would understand that indicator 65 is optional and the presently disclosed subject matter includes embodiments without such an indicator.

### VI. Product 25

The disclosed package and methods can be employed to disinfect a wide variety of products. To this end, product 25 is not limited and can include any of a wide variety of solid and/or liquid products. For example, product 25 can comprise sports equipment (pads, helmets, shoes, inserts, and the like), military gear (armor, vests, pads, helmets, and the like), fire and rescue equipment (jackets, helmets, gloves, pants, breathing masks, and the like), medical equipment, dental equipment, and so forth. One of ordinary skill in the art would appreciate that the above list is not exhaustive and that product 25 can include any of a large assortment of products.

### VII. Methods of Using the Disclosed Package

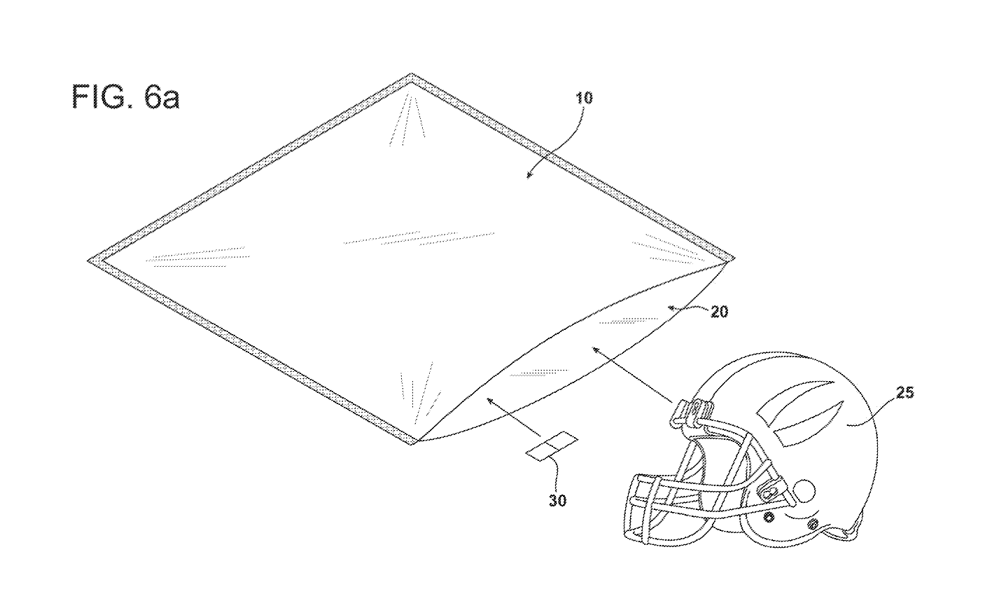

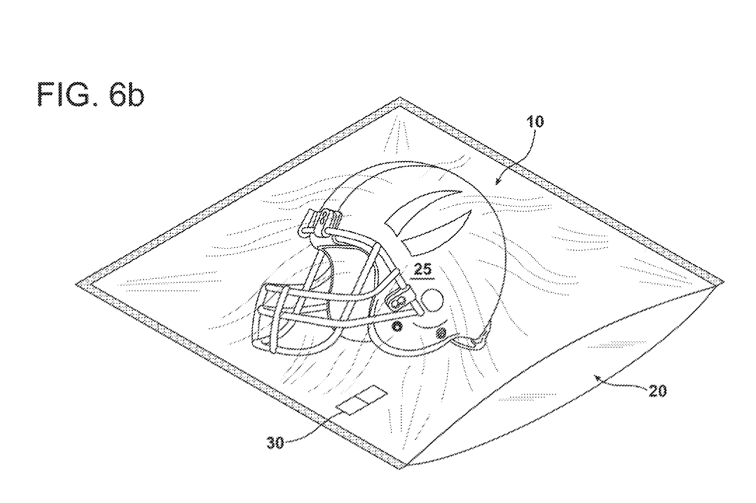

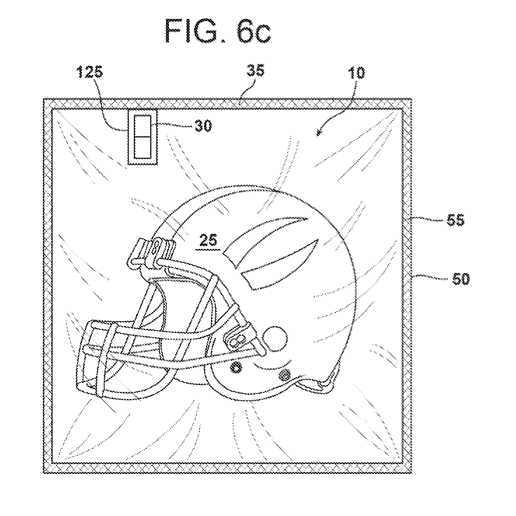

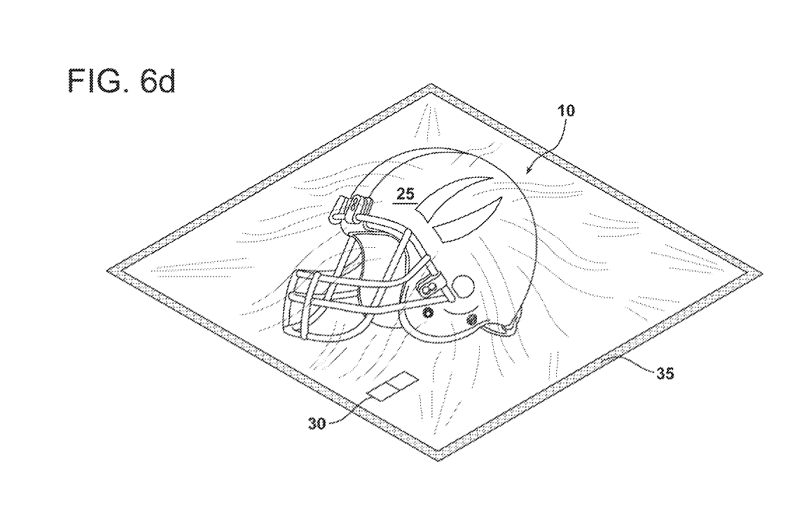

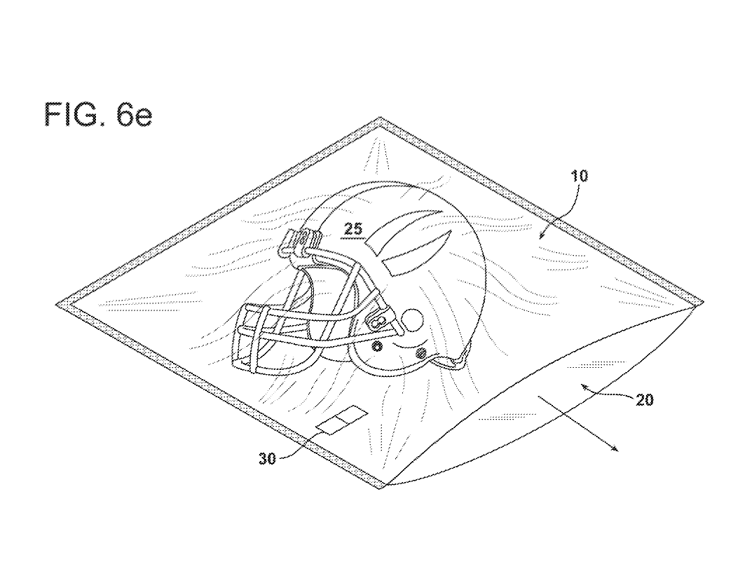

As set forth above, the presently disclosed subject matter provides a portable disinfecting package. Particularly, in some embodiments, the package includes package 10 optionally comprising at least one neutralizing layer, a disinfecting gas generator, and a product to be disinfected. In use, product 25 and generator 30 are placed into the interior of package 10 through opening 20, as illustrated in FIGS. 6a and 6b. Alternatively, in some embodiments, package 10 can be prepackaged to include generator 30. Optionally, in some embodiments, generator 30 can be contained in receptacle 125 as set forth in FIG. 6c. Generator 30 can be positioned within the interior of package 10 to allow open access to at least one emitting surface of the generator (i.e., a surface that is emitting disinfecting gas during disinfecting). Package 10 can also include indicator 65, as described herein.

Package opening 20 is then sealed, for example, by securing the opening between two overlapping margins to form seal 35, as illustrated in FIG. 6d. Seal 35 can be a heat seal, a pressure adhesive seal (reclosable or tamper-evident), a reclosable zipper-type seal, reusable clip, or any other sealing means known in the art. As will be clear from the instant disclosure, the presently disclosed subject matter is not limited to the particular type of seal used to close package 10.

Generator 30 is then activated at a desired time by the user. For example, in some embodiments, the user can apply pressure to sachet 70 through package 10 to rupture frangible seal 90 and allow reactants 80, 85 to intermix and generate the disinfecting gas. Alternatively or in addition, in some embodiments, a user can apply lid 145 to receptacle 125 to allow reactants 80, 85 to intermix and generate disinfecting gas. The activation of generator 30 depends on the particular generator used and can be accomplished through routine experimentation. Thus, in some embodiments generator 30 is activated inside package 5, such as upon closure and sealing of the package. After activation of generator 30, disinfecting gas is diffused from generator 30 into the interior of package 10 where it contacts product 25 for the duration of the disinfecting period. In some embodiments, the disinfecting period can be from 1 to 24 hours. In some embodiments, the disinfecting period can be 8 hours or less.

During this time, the disinfecting gas is gradually neutralized by the active agent present in the neutralizing layer of package 10 and/or in one compartment of generator 30. As set forth above, in some embodiments, the active agent can comprise scavenging agents that bind and thereby neutralize the disinfecting gas. Alternatively or in addition, in some embodiments, the active agent can comprise a catalyst that breaks down the disinfecting gas into components that react with a scavenging agent. One of ordinary skill in the art would understand that the above examples are not exhaustive and the disclosed active agent can include any agent that deactivates or neutralizes a disinfecting gas. The reactivity of the active agents is balanced such that the rate of neutralizing is lower than the initial rate of disinfecting gas release. As a result, the disinfecting gas has time to function fully before it is neutralized. Accordingly, when a user opens the package, exposure to the disinfecting gas is minimized or eliminated. Thus, as illustrated in FIG. 6e, at the end of the disinfecting period, a user can safely open package 10 and remove disinfected product 25 without the risk of exposure to dangerous levels of disinfecting gas.

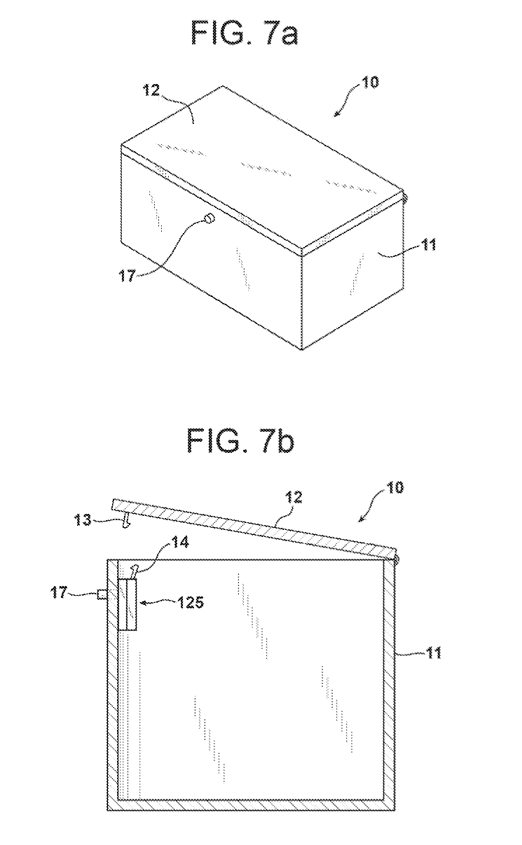

In some embodiments, package 10 can be a rigid container, as illustrated in FIGS. 7a and 7b. Specifically, package 10 can be constructed from any of a wide variety of rigid materials, including (but not limited to) plastic, metal, wood, cardboard, composite material (such as fiber-reinforced polymer) and manufactured via any commercially available process such as stamping, casting, or injection molding, and the like. In the embodiment illustrated in FIGS. 7a and 7b, package 10 includes base 11 and lid 12. In some embodiments, lid 12 is connected to base 11 via a hinge or other mechanism that allows lid 12 to open and close. In some embodiments, lid 12 can include at least one latch to secure the lid to the base. For example, in some embodiments, first latch component 13 can be positioned on lid 12 and second latch component 14 can be positioned on base 11. The latch components can include interlocking fingers that engage the lid and base when in a locked position. Thus, in some embodiments, package 10 can include a locking mechanism that functions to lock the lid to the base when in the closed position. In some embodiments, latch components 13, 14 can include a timer that delays the movement of the locking element to the locked position for a predetermined period of time such that lid 12 cannot be opened during disinfection. It should be understood that the locking component and the timer can be independent of latch components 13, 14 and are optional.

As illustrated in FIG. 7b, in some embodiments, receptacle 125 can be integrated within package 10. Specifically, receptacle can be coupled to the interior of package 10 via adhesive, a welding process, mechanical brackets, and the like. In some embodiments, activation of disinfecting gas generation of the receptacle can be controlled via actuator 17 which is accessible from the exterior of package 10. Actuator 17 can include any of a wide variety of mechanisms, including (but not limited to) valves, switches, buttons, touch plates, sliders, triggers, and the like. Thus, in use, once lid 12 has been secured onto package base 11, a user can activate activator 17 which effects a compression of sachet 70 within receptacle 125 to rupture frangible seal 90 and allow reactants 80, 85 to intermix and generate the disinfecting gas. Alternatively or in addition, in some embodiments, once activator 17 is activated, lid 145 is positioned onto receptacle 125 to allow reactants 80, 85 to intermix and generate disinfecting gas. The described latch, timer, lock, and activator mechanisms are well known in the art and thus no further detail will be given herein. See, for example, U.S. Pat. Nos. 7,290,418; 5,058,855; 5,584,682; 7,055,271; 5,986,962; 6,224,751; 5,933,391; 6,294,997; 7,033,347; 6,139,073; 6,786,519; 6,760,964; 5,086,631; 4,972,457; 5,523,730; 5,867,082; and 5,990,772, the entire disclosures of which are incorporated herein by reference.

Thus, the presently disclosed package delivers biocidal-effective amounts of a disinfecting gas (such as chlorine dioxide). The disclosed apparatus and methods achieve delivery of a desired amount of gas, at a desired rate, over a desired time period. The amount, rate and duration of delivery of disinfecting gas can be manipulated by, e.g., choice of generator, generator volume, reactant amount, reactant ratio, and the like. Such manipulations can be exercised by the artisan using only routine experimentation in view of the teachings disclosed herein together with knowledge in the art.

When package 10 is eventually opened after completion of the disinfecting period, residual disinfecting gas has been substantially eliminated from the interior of the package. In some embodiments, the concentration of disinfecting gas within the interior of package 10 at the end of the disinfecting cycle is less than 1000 ppm; in some embodiments, less than about 100 ppm; in some embodiments, less than 10 ppm; in some embodiments, less than about 1 ppm; in some embodiments, less than 0.1 ppm; and in some embodiments, less than about 0.01 ppm.

The presently disclosed subject matter also contemplates the use of kits. For example, such kits can include package 10 comprising a neutralizing layer and an opening through which the product to be disinfected can be inserted. The kit can also include generator 30 and optionally indicator 65. In some embodiments, the kit can include instructions for use. In some embodiments, package 10 and/or generator 30 can be reusable. In some embodiments, refill kits can be provided for use with the disclosed apparatus.

### VIII. Benefits of the Disclosed Package

The presently disclosed subject matter provides an assembly and methods for the controlled, on-site disinfection of a product. Employing the disclosed package, disinfecting gas can be produced safely, efficiently and economically. Moreover, the disclosed package can minimize or eliminate the release of disinfecting gas at the end of the disinfecting period.

Continuing, the disclosed package assembly is portable and can be configured such that it is able to be readily moved from place to place. Thus, the package can be assembled and disassembled quickly according to the needs of a particular user.

In embodiments wherein the disclosed package is reusable, waste can thereby be minimized. In addition, such a reusable package provides an economic benefit, as well as an environmental advantage by conserving resources.

In addition, the disclosed packages provide an attractive alternative for small volume users who may be unwilling to devote the space or resources required for using a typical autoclave or chemical disinfection system. To this end, the disclosed packages require less energy for disinfection, compared to large-scale packages and methods currently used in the art.

Further, the products to be disinfected are maintained within the interior of the packages, along with the disinfecting gas generator. Accordingly, the disclosed system requires less user handling of the package, which minimizes the propensity for breaching the disinfecting conditions.

Another advantage of the presently disclosed subject matter is the improved economy of space and packaging materials. Particularly, refill kits can provide reactants in loose or pre-measured form, without the need for delivery, storage and use of new reactant chambers containing reactant after every use. The use of pre-measured forms of reactants further allows for the convenient generation of a desired concentration or amount of gas designed for the volume of equipment to be treated.

Although several advantages of the disclosed system are set forth in detail herein, the list is by no means limiting. Particularly, one of ordinary skill in the art would recognize that there can be several advantages to the disclosed system that are not included herein.

### EXAMPLES

The following Examples provide illustrative embodiments. In light of the present disclosure and the general level of skill in the art, those of ordinary skill in the art will appreciate that the following Examples are intended to be exemplary only and that numerous changes, modifications, and alterations can be employed without departing from the scope of the presently disclosed subject matter.

### Example 1

### Preparation of Samples 1-5

Sample 1 was prepared by combining 20% by weight of 85% purity sodium chlorite (ground to a fine powdery consistency) with 80% by weight quartz sand (both available from Aldrich Chemical Company, Milwaukee, Wis., United States of America).

Sample 2 was prepared by combining 40% by weight of 85% sodium chlorite (ground to a fine powdery consistency) with 60% by weight quartz sand (both available from Aldrich Chemical Company, Milwaukee, Wis., United States of America).

Sample 3 was prepared by combining 50% by weight citric acid (provided by Aldrich Chemical Company, Milwaukee, Wis., United States of America) and 50% by weight tap water.

Sample 4 was prepared by combining 10% by weight potassium iodide (provided by Aldrich Chemical Company, Milwaukee, Wis., United States of America) and 90% by weight of tap water.

Sample 5 was prepared by combining 10% sodium sulfite (provided by Aldrich Chemical Company, Milwaukee, Wis., United States of America) and 90% by weight of tap water.

Table 1 is provided below, summarizing the preparation of samples 1-5.

TABLE 1
Summary of Samples 1-5

| | Weight Percent | | | | | |
|---|---|---|---|---|---|---|
| | Sodium | Quartz | Citric | Potassium | Sodium | |
| Sample | Chlorite | sand | Acid | Iodide | Sulfite | Water |
| 1 | 20 | 80 | — | — | — | — |
| 2 | 40 | 60 | — | — | — | — |
| 3 | — | — | 50 | — | — | 50 |
| 4 | — | — | — | 10 | — | 90 |
| 5 | — | — | — | — | 10 | 90 |

### Example 2

### Preparation of Sachets 1 and 2

Sachet 1 was prepared from a commercially available chemical-protective Tyvek® Coverall (purchased from DuPont, Wilmington, Del., United States of America), which was known to be a vapor-permeable but liquid-impermeable material. After filling Sachet 1 with about 5-10 grams of sample, the sachet was perimeter heat sealed using an impulse sealer, which uses a combination of heat and pressure to form the seal (approximately 3 second impulse durations). Sachet 1 was constructed to a size of about 2 inches by about 3 inches.

Sachet 2 was prepared from a commercially available nonwoven heat sealable teabag material, which was known to be liquid-permeable and vapor-permeable, but solid-impermeable. After filing with about 5-10 grams of sample, Sachet 2 was perimeter heat sealed using an impulse sealer, which uses a combination of heat and pressure to form the seal. Sachet 2 was constructed to a size of about 2 inches by about 3 inches.

### Example 3

### Preparation of Film 1

A 2-mil×6 inch wide monolayer film (Petrothene NA 345-013, available from LyondellBasell Polymers, Rotterdam, Netherlands) containing a 5% loading of Irganox 1076 (a commercial antioxidant available from Ciba Specialty Chemicals, Basel, Switzerland) in LDPE resin was extruded on a small extrusion line equipped with a sheet die and chill roll take up. The resultant film was referred to as Film 1.

### Example 4

### Preparation of Pouches 1 and 2

Pouch 1 was a 14 inch×18 inch nylon-containing coextruded and heat sealed pouch having a zipper seal on one side. The layer composition of Pouch 1 was LLDPE/Tie/Nylon/Tie/LLDPE and the pouch thickness was 3.5 mils. Pouch 1 had an internal gas space of about 1 gallon.

Pouch 2 was a commercially available Spacemaker® Suit Bag (available from Whitmor, Inc., Southaven, Miss., United States of America) measuring about 27 inches×41 inches and had an internal garment hanging feature and a hermetic side-zipper seal. Pouch 2 had an internal gas volume of about 10 gallons when a rectangular plastic basket was placed inside the bag to hold the walls apart near the bottom.

### Example 5

### Chlorine Dioxide Production and Scavenging Testing

A form made from two sheets of 1-inch thick closed cell polyethylene foam was placed into Pouch 1 to hold the volume of the pouch to about 1 gallon. Sachet 2 containing 5-10 grams of a scavenger candidate (solid powder) was positioned in the center of the pouch, on top of the form. The scavenger candidates tested were control (no scavenger), 10 g sodium sulfite (provided by Aldrich Chemical Company, Milwaukee, Wis., United States of America), 10 g ascorbic acid (provided by Aldrich Chemical Company, Milwaukee, Wis., United States of America), 10 g Irganox 1076 (available from Ciba Specialty Chemicals, Basel, Switzerland), and 5 g ascorbic acid (provided by Aldrich Chemical Company, Milwaukee, Wis., United States of America).

Sachet 1 was charged with 2.5 grams Sample 1 followed by 1.25 mL Sample 3. Sachet 1 was taped to the inside of Pouch 1 and the pouch was immediately closed. Chlorine dioxide gas concentration was then monitored using the syringe method described below.

Particularly, 0.2 grams of Sample 3 solution and 0.8 grams Sample 4 solution was combined immediately prior to use (“the reagent”) and taken up into a 60 cc plastic syringe. The syringe needle was then loaded into the pouch being sampled and about 50 cc of gas was taken in. The syringe was swirled for about 30 seconds before expelling the gas, taking care not to expel the reagent. The method was repeated up to 5 times to effect a readily observable color change (clear to yellow-orange) in the reagent.

The reagent was then expelled into a 1.5 mL GC vial and a visual comparison was made with a series of vials containing a serial dilution series of reference standard reagent solutions derived from a parent solution of known degree of oxidation by chlorine dioxide gas, as based on titration of the oxidized solution against Solution 5. The color standards were made without the use of Sample 5 to afford stability in their color over time by avoiding acid-induced over-oxidation.

The parts per million by volume (ppmv) level of chlorine dioxide gas in the test pouch from the color-comparison concentration level in the test reagent was calculated, allowing for the volume of the pouch and the number of 50 cc samples used to colorize the reagent.

Table 2 illustrates the chlorine dioxide profiles for the scavenger candidates.

TABLE 3
Chlorine Dioxide Profiles for Scavenger Candidates

| | Scavenger Candidates | | | | |
|---|---|---|---|---|---|
| | | 10 g | 10 g | 10 g | 5 g |
| | | Sodium | Ascorbic | Irganox | Ascorbic |
| Time | Con- | Sulfite (ppmv | Acid (ppmv | 1076 (ppmv | Acid (ppmv |
| (min) | trol | ClO₂) | ClO₂) | ClO₂) | ClO₂) |
| 15 | 7500 | 6250 | 5000 | 5000 | 11250 |
| 30 | 7500 | 6250 | 3750 | 3000 | 10000 |
| 60 | 6250 | 5000 | 2500 | 2000 | 6750 |
| 120 | 5000 | 5000 | 1250 | <1250 | 3750 |
| 170 | — | — | — | — | 2500 |
| 1080 | 3250 | 2000 | 350 | — | <100 |
| 1440 | 2000 | 750 | <25 | — | — |

The results from Table 3 indicate that chlorine dioxide gas is effectively produced by a Sample 1 and Sample 3 mixture. Of the scavengers tested, Irganox 1076 was the most effective, and sodium sulfite was the least effective. It was also noted that in the control and sodium sulfite test, a slight film formed inside the pouch wall. Rinsing with about 10 mL of water and checking the pH using pH paper yielded a pH between about 1.5 and 2.0 (highly acidic), suggesting that persistence of high levels of chlorine dioxide could lead to acidic by-product formation. In contrast, the water rinsed from the ascorbic acid and Irganox test pouches gave a pH reading of 5 or greater at the end of the test, suggesting that the scavenger was serving to prevent the acidic residue from forming.

### Example 6

### Chlorine Dioxide Production and Scavenging

A Sunon 5V DC 0.35 Watts electric fan (available from Sunonwealth Electric Machine Industry Co., Ltd. of Taiwan) was obtained. The fan was powered by a 6×AAA NiMH rechargeable battery pack mounted inside a plastic housing designed to hold the fan and battery as well as one or more sachets, and to circulate air past one surface of a sachet. Two types of air circulation holders were employed. One type (designated Fan 1) contained a single fan and a single sachet chamber. Fan 1 was positioned to circulate air through only the sachet chamber.

A Fan 1 circulator was positioned in the center of Pouch 1. Sachet 2 containing 5-10 grams of a scavenger candidate (solid powder) was positioned in the Fan 1 circulator chamber, which was on the intake side of the fan. The scavenger candidates tested were control (5 g ascorbic acid) and 5 g sodium d-iso-ascorbate (provided by Aldrich Chemical Company, Milwaukee, Wis., United States of America). The chlorine dioxide gas concentration was monitored using the syringe method described in Example 5. The fan was turned on immediately after collecting the first gas sample at 15 minutes. Results are given below in Table 4.

TABLE 4
Chlorine Dioxide Concentration Profiles for Scavenger Candidates

| | Contents of Scavenger Test Sachet in Chamber of Fan 1 | |
|---|---|---|
| | 5 g Ascorbic Acid | 5 g Sodium d-iso-Ascorbate |
| Time (min) | (ppmv ClO₂) | (ppmv ClO₂) |
| 15 | 6250 | 6250 |
| 30 | 2250 | 625 |
| 45 | — | 100 |
| 60 | 425 | 50 |
| 90 | 125 | 20 |
| 120 | 50 | 17 |
| 155 | 43 | — |
| 185 | 35 | — |

Table 4 indicates that sodium d-iso-ascorbate is an extremely effective chlorine dioxide scavenger when used as a solid. It also shows, by comparison with the two 5 g ascorbic acid tests across Example 5, that fan-assisted air circulation is extremely beneficial to scavenging. For example, Example 5 showed that at 120 minutes without a fan there was a reading of 3750 ppmv chlorine dioxide. The corresponding level with the fan assist at 120 minutes was 50 ppmv.

### Example 7

### Air Circulation Fans Setup

A Sunon 5V DC 0.35 Watts electric fan (available from Sunonwealth Electric Machine Industry Co., Ltd. of Taiwan) was obtained. The fan was powered by a 6×AAA NiMH rechargeable battery pack mounted inside a plastic housing designed to hold the fan and battery as well as one or more sachets, and to circulate air past one surface of a sachet. Two types of air circulation holders were employed. One type (designated Fan 1) has been described above in Example 6. The second type (designed Fan 2) contained two fans (F1 and F2) on a three-position switch to activate one fan at a time and two compartments (C1 and C2) for containing sachets. Fan F2 was positioned within Pouch 2 to circulate air through C1 and C2. C1 served as the overall air exit for the Fan 2 holder, whereas C2 served as a pre-chamber that exited into C1.

Sachet 1 was charged with 2.5 grams Sample 1 followed by 1.25 mL Sample 3. The sachet was positioned near the air exit of the Fan 2 device. The Fan 2 device was hung from a garment hook near the top of Pouch 2, and a plastic basket was positioned in the bottom of the pouch to enforce an approximately 10 gallon internal volume. The Fan 2 device contained a scavenger pouch inside compartment C2. The scavengers tested included control (no scavenger), 5 g sodium d-iso-ascorbate in Sachet 1, and 5 g sodium d-iso-ascorbate in Sachet 2. The Fan 2 device provided a means to circulate air without circulating it past the scavenger sachet, then to switch over and circulate the air past the scavenger sachet. Results are given below in Table 5.

TABLE 5
Chlorine Dioxide Concentration Profiles
for Scavenger Candidates with Fan 2

| | Contents of Scavenger Test Sachet in Chamber 2 of Fan 2 | | |
|---|---|---|---|
| | (ppmv ClO₂) | | |
| | | 5 g Sodium d-iso- | 5 g Sodium d-iso- |
| | | ascrobate in Sachet | ascorbate in Sachet |
| Time (min) | Control | 1 | 2 |
| | Fan F1 turned on, Fan F2 turned off | | |
| 0 | — | 250 | — |
| 10 | — | — | — |
| 15 | 250 | — | 240 |
| 20 | Fan F1 turned off, Fan F2 turned on | | |
| 30 | 280 | 300 | 240 |
| 50 | — | 300 | — |
| 60 | 300 | — | 180 |
| 90 | — | — | 130 |
| 100 | — | 180 | — |
| 120 | 260 | — | 80 |
| 150 | — | — | 50 |
| 165 | — | 120 | — |
| 180 | — | — | 25 |
| 210 | — | — | 15 |
| 245 | — | 70 | — |
| 280 | 200 | — | — |
| 310 | — | 40 | — |
| 360 | 120 | — | 3 |

Table 5 demonstrates that the Fan 2 alone does not significantly reduce the chlorine dioxide concentration. The use of a scavenger in Sachet 2 proved to be much more effective, reducing the chlorine dioxide concentration to less than 5 ppmv within 6 hours.

### Example 8

### Chlorine Dioxide Profile for Integrated Sachet Construction

2.5 grams Sample 1 was heat sealed inside Sachet 2 material. 2.0 grams Sample 3 was heat sealed inside a pouch constructed from a multilayer plastic film (RV 335 MZ frangible seal film, available from Packall Packaging, Inc., Brampton, Ontario) having a specialized heat seal layer with a controlled low bond strength, for producing frangible seals. 5.0 grams sodium-d-iso-ascorbate was heat sealed inside a one-compartment or two-compartment sachet constructed from Sachet 2 material. The purpose of the 2 compartment design was to increase the exposed surface area of the solid scavenger to maximize the reactivity at the air interface.

An outer sachet about 2 inches×3.5 inches was constructed by sealing a layer of plastic multilayer film from Pouch 1 against a layer of Sachet 1 material. A partial inner seal was made to divide the sachet into two equal compartments that allow liquid to move between them using an impulse sealer. The Sample 1 and Sample 3 sachet and pouch, one in each chamber, and the outer edge was fully sealed using an impulse sealer.

The sodium-d-iso-ascorbate sachet was affixed to the Sample 3 end of the outer sachet to extend beyond the outer sachet's length, giving an overall master sachet assembly about 2 inches×6 inches in size. The master sachet assembly was inserted into a Fan 2 holder designed such that when the master sachet was inserted, the sodium-d-iso-ascrobate sachet was housed in chamber C2 and the combination sachet with Sample 1 and Sample 3 was housed in adjoining chamber C1. Air flow paths were present along side all sachets. In chamber C1, the permeable side of the sachet (Sachet 1 material) was oriented towards the air circulation zone of that chamber.

The Fan 2 holder with enclosed master sachet assembly was hung inside Pouch 2 as in Example 7. The Sample 1 portion was oriented at the bottom end. After closing Pouch 2 at the start of the test, a constriction zone in chamber C1 was manually compressed to burst the sample 1 pouch inside the master sachet and cause the contained liquid to run into the lower portion of the master sachet, saturating the Sample 3 material with Sample 1.

TABLE 5
Chlorine Dioxide Concentration Profiles for Three
Repeat Tests Using Integrated Master Sachet Construction
and Two Fan Air Circulation

| | 2-Compartment | 2-Compartment | 1-Compartment |
|---|---|---|---|
| Time (min) | Sachet | Sachet | Sachet |
| 0 | Fan F1 turned on, Fan F2 turned off | | |
| 10 | 80 | 120 | 114 |
| 20 | 90* | 160 | 120 |
| 30 | 74 | 140* | 116* |
| 45 | 60 | 90 | 92 |
| 60 | 50 | 70 | 80 |
| 95 | 26 | — | — |
| 100 | — | 26 | 50 |
| 130 | 12 | — | 26 |
| 135 | — | 15 | — |
| 160 | 7.5 | — | 15 |
| 165 | — | 8 | — |
| 190 | 5 | — | 7 |
| 195 | — | 4 | — |
| 220 | — | — | 5 |

*Fan F1 turned off and Fan F2 turned on after this sample.

Table 5 illustrates the comparable results obtained with 3 trials using the integrated sachet design. The data demonstrates the use of the frangible seal pouch, burst by a constriction feature on the Fan 2 holder, for mixing of reactants at the outset. The somewhat lower initial concentration of chlorine dioxide is of uncertain origin. The volume inside Pouch 2 was not strictly controlled from test to test, which could explain a large amount of the variation.

### Example 9

### Chlorine Dioxide Concentration Profiles

A series of tests was performed using the integrated master sachet and Fan 2 apparatus as described in Example 8. In each of these tests, an article of clothing was included inside Pouch 2 (army fatigue pants on a clothes hanger hung from the top of the pouch interior). Sample 2 was tested instead of Sample 1 in some cases, as set forth in Table 6 below. In some of the trials, a spore strip was included to determine antimicrobial effectiveness. The results are given in Table 7 below.

Specifically, B. Atrophaeus spore test strips were obtained from NAMSA (Northwood, Ohio, United States of America) having 1.7×10⁶ spores per strip. The strips were held at ambient temperatures prior to use. For gas exposure testing, a single test strip was placed inside a 15 mL glass screw top vial with the top removed. At the end of the gas exposure test, the top was replaced over the vial, taking care to touch only the outside surfaces of each. The vials were submitted to a test lab for determination of viable organism count in the enclosed strip.

TABLE 6
Chlorine Dioxide Profiles and Spore Strip Data

| | | | | | Sample 2 |
|---|---|---|---|---|---|
| | | Sample 1 | | Sample 2 | (spore |
| | Sample 1 | (spore | Sample 2 | (spore | strip in |
| | (no spore | strip at | (no spore | strip in | pants |
| | strip) | bottom) | strip) | bottom) | pocket) |
| Time (min) | Fan F1 turned on, Fan F2 turned off | | | | |
| 10 | — | — | 150 | — | — |
| 15 | 110 | 66 | — | 200 | 76 |
| 20 | — | — | 160 * | — | — |
| 30 | 90 * | 60 * | — | 160 * | 76 * |
| 45 | 70 | — | — | — | — |
| 55 | — | — | — | — | 48 |
| 60 | 50 | — | — | 70 | — |
| 70 | * | — | 80 | — | — |
| 90 | * | — | — | — | — |
| 95 | * | — | — | 30 | — |
| 120 | 26 | — | — | — | — |
| 125 | — | — | 40 | — | 16 |
| 150 | — | — | — | 14 | — |
| 185 | — | 3 | — | — | — |
| 205 | — | — | — | 6 | — |
| 245 | — | — | 10 | — | — |
| 270 | 4 | — | — | — | — |
| 295 | — | — | — | 2 | — |
| 300 | — | 1.5 | — | — | — |
| 320 | — | — | 2 | — | — |
| Spore Strip | N/A | >10⁶ | N/A | >10⁶ | 3.6 × 10⁴ |
| Result | | reduction | | reduction | reduction |
| | | (Sterile) | | (Sterile) | |

* Fan F1 turned off and Fan F2 turned on after this sample

The Table 6 data establish the ability to sustain high chlorine dioxide levels even with a garment in the bag, as in the intended application. It is possible that high ambient humidity played a role in the lower initial chlorine dioxide reading observed in the last column in the table (the ambient humidity for that test was about 67%, while for the other tests it was closer to 50%). The reason for this effect can be that the garment is more effective at adsorbing chlorine dioxide into its surface when in a humid environment. This experiment verifies strong antimicrobial effectiveness at the chlorine dioxide levels being produced in these tests, even when the spore strip is hidden with a pants pocket (last column in table).

### Example 10

### Chlorine Dioxide Profile for Testing Film-Based Scavenger

Film 1 was used to line the interior of Pouch 2 containing an empty plastic basket in the bottom. The lining process was performed by folding four 6-foot strips of Film 1 at the middle and hanging them across the top of the pouch interior in a way that afforded air access to both sides. A Fan 2 holder containing a sachet that included Sample 1 and Sample 3 and no scavenger was inserted into Pouch 2. Pouch 2 was then closed, and the lid was compressed to burst the Sample 3 pouch and mix Samples 3 and 1. Fan F1 was turned on and left on through the duration of the test. The results are given in Table 7 below. As indicated in the table, the film based scavenger performed very well, giving a chlorine dioxide level change from over 180 ppmv to below 1 ppmv in a period of four hours.

TABLE 6
Chlorine Dioxide Profiles and Spore Strip Data

| | Sample 1 with Scavenger Film Liner |
|---|---|
| Time (min) | (ppmv) |
| 5 | 180 |
| 15 | 186 |
| 30 | 60 |
| 60 | 24 |
| 120 | 7 |
| 180 | 3 |
| 240 | <1* |

*An electronic meter reading (CanarySense BW GasAlert) was taken inside the Pouch 10 after opening the pouch to remove the scavenging films. The reading was between 0.1 and 0.2 ppmv ClO₂

What is claimed is:

1. A container for disinfecting a product, said container comprising:
a. a package having an interior and a sealable opening through which said product can be placed into the interior of the package;
b. a disinfecting gas generator disposed within the interior of said package;
wherein said package comprises a pair of opposing films joined together along a pair of opposing sides and a bottom bridging the sides; and
wherein said package comprises at least one of the following: (1) at least one of said package films comprises a neutralizing layer and an active agent carried by the neutralizing layer, (2) an active agent housed in said disinfecting gas generator, or (3) both (1) and (2).

2. The container of claim 1, wherein said disinfecting gas is selected from the group comprising: chlorine dioxide, ethylene oxide, sulfur dioxide, nitrogen dioxide, nitric oxide, nitrous oxide, carbon dioxide, hydrogen sulfide, hydrocyanic acid, dichlorine monoxide, ozone, and combinations thereof.

3. The container of claim 1, wherein said generator is a sachet housing two reactants separated by a frangible seal, such that when the frangible seal is ruptured and the reactants are combined, disinfecting gas is generated.

4. The container of claim 3, wherein said sachet further comprises a compartment housing an active agent.

5. The container of claim 1, wherein said generator is a sachet housing two reactants and an active agent, and wherein at least one reactant is housed in a package comprising a frangible seal.

6. The container of claim 1, wherein said generator is housed in a receptacle comprising at least one fan.

7. The container of claim 1, further comprising a product disposed within the interior of the package.

8. The container of claim 1, further comprising an indicator disposed within the interior of the package, said indicator adapted to indicate when an effective amount of disinfecting gas has been generated.

9. The container of claim 1, wherein said pouch comprises a locking mechanism.

10. A method of disinfecting a product, said method comprising:
a. supplying a product to be disinfected;
b. supplying a package having an interior and a sealable opening through which said product can be placed into the interior of the package;
c. supplying a disinfecting gas generator;
d. placing said product and said generator within the interior of said package;
e. sealing said package;
f. activating said generator to introduce an amount of disinfecting gas into said interior effective to disinfect said product;
g. maintaining said package in a sealed condition for a disinfecting period;
h. opening said package and removing said disinfected product;
wherein said package comprises a pair of opposing films joined together along a pair of opposing sides and a bottom bridging the sides; and
wherein said package comprises at least one of the following: (1) at least one of said package films comprises a neutralizing layer and an active agent carried by the neutralizing layer, (2) an active agent housed in said disinfecting gas generator, or (3) both (1) and (2).

11. The method of claim 10, wherein the concentration of disinfecting gas is sufficient to disinfect said product.

12. The method of claim 10, wherein said disinfecting period is about 8 hours or less.

13. The method of claim 10, wherein said disinfecting period is about 6 to 24 hours.

14. The method of claim 10, wherein said concentration of disinfecting gas within the interior of said package at the end of said disinfecting period is less than about 10.0, 1.0, 0.1, or 0.01 parts per million.

15. The method of claim 10, wherein said disinfecting gas is selected from the group comprising: chlorine dioxide, ethylene oxide, sulfur dioxide, nitrogen dioxide, nitric oxide, nitrous oxide, carbon dioxide, hydrogen sulfide, hydrocyanic acid, dichlorine monoxide, ozone, and combinations thereof.

16. The method of claim 10, wherein said generator is a sachet housing two reactants separated by a frangible seal, such that when the frangible seal is ruptured and the reactants are combined, disinfecting gas is generated.

17. The method of claim 10, wherein said sachet further comprises a compartment housing an active agent.

18. The method of claim 10, wherein said generator is a sachet housing two reactants and an active agent, and wherein at least one reactant is housed in a package comprising a frangible seal.

19. The method of claim 10, wherein said generator is housed in a receptacle comprising at least one fan.

20. The method of claim 10, further comprising a product disposed within the interior of the package.

21. The method of claim 10, further comprising an indicator disposed within the interior of the package, said indicator adapted to indicate when an effective amount of disinfecting gas has been generated.

22. The method of claim 10, wherein said pouch comprises a locking mechanism.

23. A kit comprising:
a. a package having an interior and a sealable opening;
b. a disinfecting gas generator;
wherein said package comprises a pair of opposing films joined together along a pair of opposing sides and a bottom bridging the sides; and
wherein said package comprises at least one of the following: (1) at least one of said package films comprises a neutralizing layer and an active agent carried by the neutralizing layer, (2) said disinfecting gas generator comprises an active agent, or (3) both (1) and (2).

24. The kit of claim 23, wherein said disinfecting gas is selected from the group comprising: chlorine dioxide, ethylene oxide, sulfur dioxide, nitrogen dioxide, nitric oxide, nitrous oxide, carbon dioxide, hydrogen sulfide, hydrocyanic acid, dichlorine monoxide, ozone, and combinations thereof.

25. The kit of claim 23, wherein said generator is a sachet housing two reactants separated by a frangible seal, such that when the frangible seal is ruptured and the reactants are combined, disinfecting gas is generated.

26. The kit of claim 23, wherein said neutralizing layer is positioned directly adjacent to a permeable layer.

27. The kit of claim 23, further comprising an indicator disposed within the interior of the package, said indicator adapted to indicate when an effective amount of disinfecting gas has been generated.
